(19) United States
(12) Patent Application Publication
(10) Pub. No.: US 2002/0138078 A1
(43) Pub. Date: Sep. 26, 2002

(54) System and method for cutting grafts

(76) Inventor: James L. Chappuis, Marietta, GA (US)

Correspondence Address: THOMAS, KAYDEN, HORSTEMEYER & RISLEY, LLP, 100 GALLERIA PARKWAY, NW, STE 1750, ATLANTA, GA, 30339-5948, US

(21) Appl. No.: 09/814,202

(22) Filed: Mar. 21, 2001

(51) Int. Cl. A61B 17/14

(52) U.S. Cl. 606/85

(57) ABSTRACT

A system and method to facilitate bone grafting, the system including a rasp configured to prepare the grafting surfaces of a recipient, a sizer configured to determine the dimensions of a graft for abutment with the grafting surfaces, a cutting guide configured to receive bone stock and to facilitate producing the graft in proportion to the sizer, a measuring pan configured to determine the amount of grafting substance required to fill the void in the graft, and a holding device configured to stabilize the graft as the grafting substance is placed in the graft.

### BACKGROUND OF THE INVENTION

1. Technical Field

The present invention is generally related to systems, devices, and methods for producing surgical interbody segments, and more particularly to producing grafts for fusion applications.

2. Description of the Related Art

Bone grafting may be required for any number of reasons, for example, bone fractures with bone loss, repair of bone that has not properly healed, and treatment of joints to prevent movement (fusion). Of these, fusion of spine segments, or vertebral bodies is generally the most recognized by the public. Most often, an intervertebral body, or disk, is damaged, degenerates, or otherwise becomes diseased, causing great discomfort by way of impinging on the spinal cord or nerve roots. When more conservative treatments and minimally invasive procedures have been exhausted, it may become necessary to surgically remove the damaged disk and fuse the associated vertebral bodies in order to restore the original spacial relationships, as well as desired stability.

Once the damaged disk has been removed, a bone segment shaped similarly to the disk is placed in the intervertebral space to facilitate spinal fusion. Presently, these bone segments are produced in the desired dimensions from larger segments of bone, or bone stock, by medical suppliers, at great cost. Currently, practitioners have access to bone stock, which is considerably less expensive than the individually produced bone grafts on a cost per unit weight basis. It therefore follows that a practitioner could help to keep the overall cost of the fusion procedure down by producing the individual bone graft to be used from bone stock.

Thus, there is a need for improved devices, systems, and/or methods that address these and/or shortcomings of the prior art.

### SUMMARY OF THE INVENTION

The present invention relates to a system and method for facilitating bone grafting. A preferred embodiment of the system includes a rasp configured to prepare the grafting surfaces of a recipient. A sizer is also provided that is configured such that the dimensions of the graft that will abut and eventually connect both grafting surfaces may be determined. After determining the required dimensions of the graft, a cutting guide configured to receive a piece of bone stock is used to produce the graft in proportion to the sizer. Prior to insertion of the graft into the recipient, a measuring pan is used to determine the amount of grafting substance removed from the recipient and required to fill the void in the graft. Finally, a holding device is used to stabilize the graft as the grafting substance is packed into the void of the graft.

Other systems, methods, features, and advantages of the present invention will be or become apparent to one with skill in the art upon examination of the following drawings and detailed description. It is intended that all such additional systems, methods, features, and advantages be included within this description, be within the scope of the present invention, and be protected by the accompanying claims.

### BRIEF DESCRIPTION OF THE DRAWINGS

The invention can be better understood with reference to the following drawings. The components in the drawings are not necessarily to scale, emphasis instead being placed upon clearly illustrating the principles of the present invention. Moreover, in the drawings, like reference numerals designate corresponding parts throughout the several views.

FIG. 1 is a schematic of a graft positioned between two vertebral bodies.

FIG. 1A is a top perspective view of a graft as would be used to fuse the vertebral bodies shown in FIG. 1.

FIG. 2 is a top perspective view of a preferred embodiment of a rasp 210 of the present invention as used to prepare the bone segments shown in FIG. 1 for grafting.

FIG. 3 is a top perspective view of a preferred embodiment of a sizer of the present invention as used to determine the required dimensions for the graft shown in FIG. 1.

FIG. 4 is a top perspective view of a preferred embodiment of a cutting guide of the present invention as used to produce the graft shown in FIGS. 1 and 2 in the proper dimensions.

FIG. 5 is a top perspective view of a preferred embodiment of a measuring pan of the present invention as used to determine the required amount of grafting material for use in the graft shown in FIGS. 1 and 2.

FIG. 6 is a top perspective view of a preferred embodiment of a holding device of the present invention as used to immobilize the graft shown in FIG. 1 for packing with grafting material.

### DETAILED DESCRIPTION OF THE PREFERRED EMBODIMENT

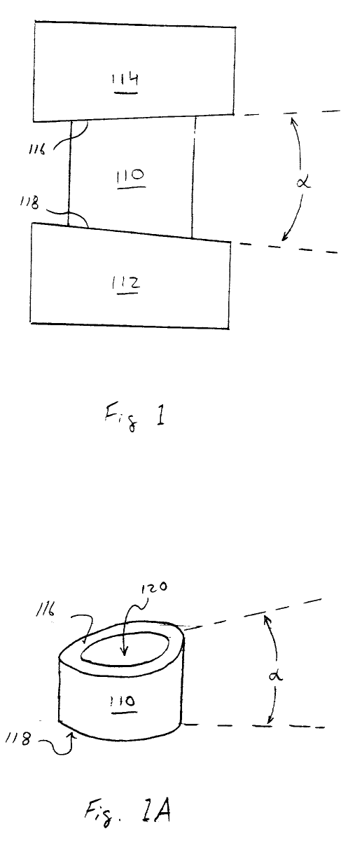

Turning now to the drawings, FIG. 1 is a schematic representation of a graft 110 positioned between two vertebral bodies 112, 114 to be fused. Although grafting may be used to fuse any number of bones, for simplicity in describing the present invention, only the fusion of two adjacent vertebrae will be discussed.

At present, once a decision has been reached that fusion of two adjacent vertebral bodies 112, 114 is required, the affected disk is removed and means are provided for causing a rigid union of the two vertebral bodies 112, 114. Small pieces of bone, usually harvested from the pelvic bone of the patient, or autograft bone, are inserted into the intervertebral space formed by removal of the disk. Eventually, these pieces of autograft bone form one mass thereby fusing the two vertebral bodies 112, 114 together. During the period of bone growth and ultimate fusion, it is desirable to stabilize the autograft bone mass to facilitate fusion. Providing support for the autograft bone may be accomplished using biologic material, meaning bone grafts, or fusion cages. Fusion cages are man-made structures, generally of metals such as titanium, that serve primarily the same functions as do bone grafts. Although some aspects of various embodiments of the present invention will not apply to the use of fusion cages, other aspects will and these will be pointed out. However, all aspects apply to the use of bone grafts, and therefore, grafting using biologic material will be discussed primarily.

The graft 110, as shown in FIG. 1, is the end product of the fusion process. As previously noted, first, the damaged intervertebral body, or disk, is removed. Once this has occurred, an intervertebral space will be left between the two vertebral bodies 112, 114 to be fused. In the fusion process, it is desirable to use a graft 110 that has substantially the same dimensions as the disk that has been removed, but has a slightly greater height dimension relative to a longitudinal axis of the spinal column. This helps ensure adequate contact is maintained between the mating surfaces 116, 118 of the graft 110 and the upper surface 113 and lower surface 115 of the first vertebral body 112 and the second vertebral body 114, respectively.

As would be expected, all disks are not similarly shaped, and therefore, all grafts 110 will not be similarly shaped. FIG. 1A shows the general configuration of a bone graft 110. Note that although the upper mating surface 116 and lower mating surface 118 of the graft 110 maybe parallel, they may also, and in most cases do, form an angle therebetween. The desired angle between the planes containing the upper and lower mating surfaces 116, 118 of the bone graft 110 is referred to as α. Also note that the graft 110 has a hollow center, or void 120. Prior to placing the graft 110 between the vertebral bodies 112, 114, grafting substance, or autograft bone, will be placed inside the void 120 to stimulate fusion.

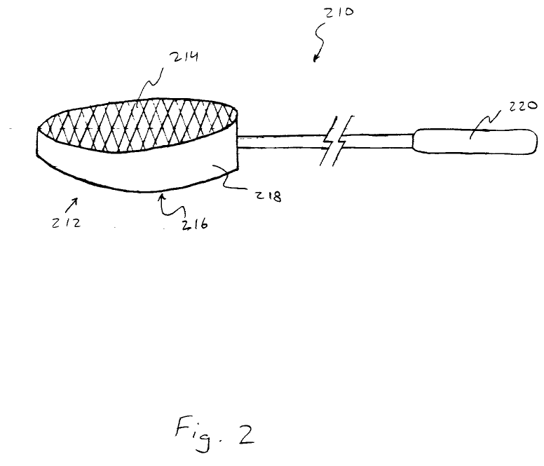

After the disk has been removed, the exposed surfaces of the upper and lower vertebral bodies 112, 114 are prepared to facilitate fusion. It is desirable to provide a roughened surface into which fusion may occur. As such, the exposed surfaces of the vertebral bodies 112, 114 are prepared with a rasp 210 as shown in FIG. 2. A preferred embodiment of the rasp 210 includes a head portion 212 with a first surface 214 and a second surface 216 configured to prepare the exposed surfaces. As such, serations, cross-hatching, or other textures are provided on the first and second surfaces 214, 216. Note, however, that it is desirable to have the intermediate surface 218 of the head 212 smooth so as not to inadvertently damage any nerve or spinal tissue while preparing the exposed surfaces of the vertebral bodies 112, 114.

Although the preferred embodiment shown in FIG. 2 teaches an angle (α) between the first and second surfaces 214, 216 similar to the angle of the intervertebral space, the first and second surfaces 214, 216 may be parallel. However, the surfaces having the angle between them offer the added advantage of preparing both vertebral bodies 112, 114 simultaneously. Note the handle 220 may be positioned as best facilitates use of the rasp 210. This should, in all likelihood, be where the intermediate surface 218 is at its greatest height.

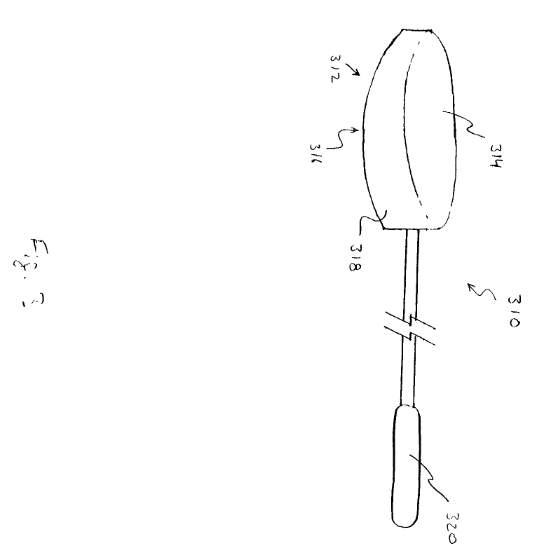

After the surfaces of the vertebral bodies 112, 114 have been prepared, it then becomes necessary to determine the dimensions of the required graft 110. In a preferred embodiment of the present invention, a plurality of sizers 310 (FIG. 3) are used for this measurement. Each sizer 310 includes a body 312, having a primary surface 314, a secondary surface 316, and a mid surface 318, the body 312 being shaped substantially like a graft 110. Depending on what type of grafting is being performed and on what portion of the body, the size of the necessary graft 110 could vary greatly. Likewise, a wide variety of sizers 310 will be required. However, for ease of description, a potential set of sizers 310 for use between vertebral members of the lumbar region will be used.

In the lumbar region, grafts 110 having a height dimension of, for example, anywhere from 10 mm to 20 mm may be required. Therefore, a proper range of sizers 310 would possibly range from 8 mm in height to 22 mm in height, at the widest point. As well, in one embodiment, the sizers 310 increase in 1 mm increments until the largest size is reached. Ideally, there would also be a number of sizers 310 having varying angles between the planes containing the primary surface 314 and the secondary surface 316 to account for the varying shaped intervertebral spaces (FIG. 1A). Note that for use in the spinal column, it has been determined that the most desirable range for α is approximately 5-9°, although various other angles may be utilized and are considered well within the scope of the present invention.

In use, a practitioner would place the body 312 of the sizer 310 into the intervertebral space. A handle 320 of appropriate length is provided so that the sizer 310 may be inserted anteriorly. Sizers 310 of increasingly larger dimensions are placed in the intervertebral space until a good fit is determined. Once a properly sized and angled sizer 310 is found, these dimensions may be used to help determine the most advantageous dimensions for a graft 110.

As previously discussed, a graft 110 having height dimensions slightly greater than the height of the intervertebral space is desired because this helps to ensure adequate contact between the mating surfaces 116, 118 of the graft 110 and exposed surfaces of the vertebral bodies 112, 114. As well, a “snug” fit will aid in preventing potential motion of the graft 110 that could hinder the fusion process. Therefore, for instance, if the sizer 310 found to properly fit the intervertebral space was 16 mm with a value of α equal to 7°, a graft 110 with a height of 18 mm and a value of α equal to 7° would be desired.

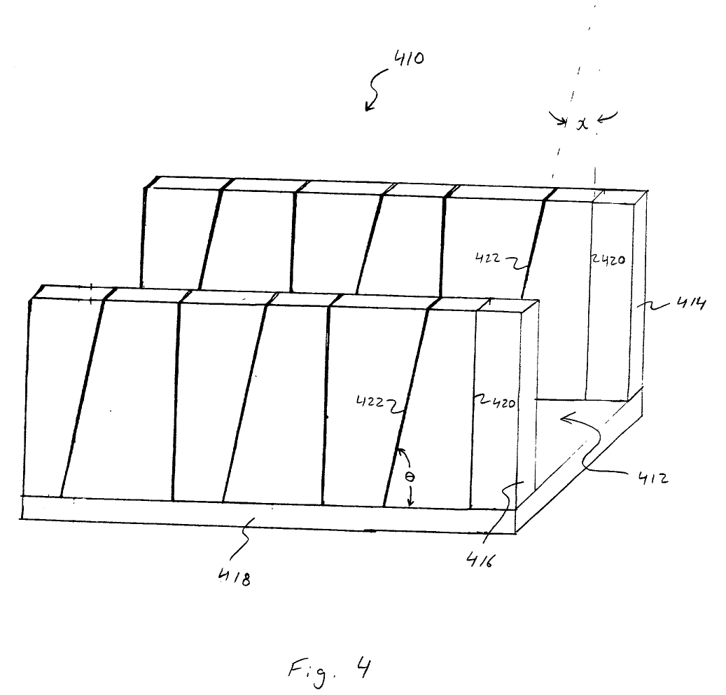

The next step in preparing the graft 110 includes cutting the graft 110 from bone stock, or other suitable material, using the desired height and angle determined previously. In so cutting the bone stock, a device like a sagital saw (not shown) will be used. To facilitate this process, a cutting guide 410 (FIG. 4) is used. Although a preferred embodiment of the cutting guide 410 is shown in FIG. 4, numerous other embodiments and configurations are possible. For example, first, the bone stock (not shown) is placed in the cavity 412 between the first and second guide structures 414, 416. Because the diameter of bone stock used will vary depending on the diameter of the graft 110 required, the embodiment shown discloses the maximum graft height measurement as being the portion of the bone stock that is in contact with the base 418. Note, however, that the maximum graft height could be marked along the top of the guide structures, so long as a spacer or similar object were placed between the bone stock and the base 418 such that the edge of the bone stock was made flush with the plane containing the tops of both the first guide structure 414 and second guide structure 416.

After the bone stock has been placed in the cavity 412, it is held securely in place by any adequate means. Once secured, the graft 110 is cut from the bone stock using the sagital saw, or similar device, the guide slots providing the proper dimensions. Note that the configuration of guide slots shown in FIG. 4 is only one of many possibilities. For example, as shown, the first guide slot 420 is perpendicular to the longitudinal axis of the base 418 while the second guide slot 422 forms an angle θ with the base 418 that is the compliment of the angle α, α being the angle at which the planes will ultimately intersect. However, the guide slots could also be configured such that both guide slots intersect the base 418 at equivalent angles such that the graft 110 is divided into identical halves by the line bisecting the angle α. As well, another embodiment could include a top connecting the guide structures such that an enclosure is formed, rather than a cavity 412, into which the bone stock is placed.

After the properly sized graft 110 has been produced, grafting material (not shown) is packed into the interior void 120 of the graft 110 (FIG. 1A). The grafting material serves to fuse the adjacent vertebral bodies 112, 114 together. The grafting material is generally taken from the pelvic bone of the patient receiving the graft 110. More specifically, bone fragments removed form the iliac crest of the pelvis are used as the grafting material. As such, there is a vital interest in removing only as much of the grafting substance as is needed for the grafting process. Also, when fusion cages are used in the grafting process vice biologic grafts, they are similarly packed with grafting material removed from the patient. Therefore, similar concerns exist when using either biologic grafts or fusion cages.

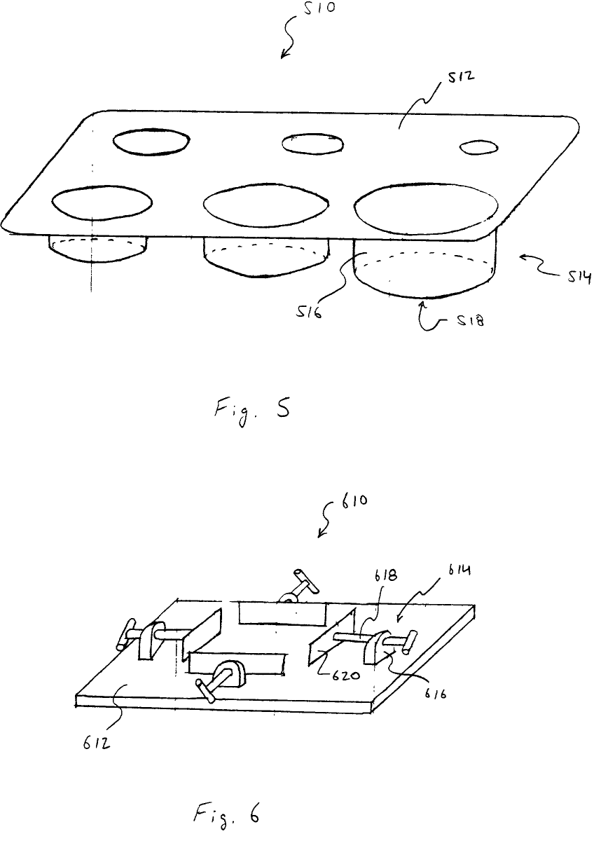

As shown in FIG. 5, a bone graft measuring pan 510 aids the practitioner in determining the amount of grafting material required to fill the void 120 in the graft 110. In a preferred embodiment, the bone graft measuring pan 510 includes a planar surface 512 in which a plurality of graduated cups 514 have been formed. Although not required, as shown, each cup includes a cylindrical side wall 516 and a flat bottom 518. So configured, the cup resembles the void 120 in either the graft 110 or the fusion cage, and therefore aids the practitioner in estimating the amount of grafting substance that needs to be harvested from the patient. Also, as the grafting substance is harvested, it may be placed in the correspondingly sized cup 514 to determine the volume of each piece harvested. In this manner the total amount of grafting substance harvested from the patient may be tracked. This aids the practitioner in determining when it may be wise to move to another harvesting site on the patient. This should prevent over harvesting and decrease morbidity in the area from which the grafting substance is taken.

Although the embodiment of the bone graft measuring pan 510 shown in FIG. 5 reveals cups 514 shaped similarly to the void 120 of a graft 110 or fusion cage, the present invention encompasses cups 514 of any shape. Ideally, the cups 514 will be graduated in increments, such as 2.5 cc or 5 cc, to aid the practitioner. Also, it may prove useful to have the bottom 518 of the cups 514 all in one plane, that plane being parallel to planar surface 512. In that way, the bone graft measuring pan 510 will remain stationary on a flat surface. If this is not desired, legs (not shown) or other similar structures may be provided on the corners of the planar surface 512 to enhance stability. Also, the bone graft measuring pan 510 can be manufactured of any suitable substance, to include disposable materials such as plastics.

While packing the graft 110 with the grafting substance, it is necessary to immobilize the graft 110. Therefore, a holding device 610 preferably is provided. A preferred embodiment of the holding device, as shown in FIG. 6, includes a planar support 614 for receiving the graft 110 and a plurality of adjustment devices sufficient to secure grafts 110, or fusion cages, of various sizes. As shown, each adjustment device 614 is attached to the planar support 612 by an adjustment support 616 configured to receive a threaded adjustment member 618. The inner end of the adjustment member 618 is configured to receive a plate 620 for abutting the graft 110 or fusion cage. However, the inner end of the adjustment member 618 could be used to abut the graft 110, thereby negating the need for the plate 620. The outer end of the adjustment member 618 includes a handle 622 or like device to facilitate securing the graft 110 with the adjustment device.

Note that while the embodiment shown in FIG. 6 discloses four adjustment devices 614, an embodiment including only one adjustment device 614 may provide adequate flexibility for handling variously sized grafts 110. A possible embodiment using only one adjustment member 618 could include an abutment member (not shown) against which the sidewall of the graft 110 would be placed. The abutment member could be an angled wall portion or similar structure that was sufficient to prevent the graft 110 from moving laterally as the adjustment device 614 is used to secure the graft 110 in place. Also, other embodiments of adjustment devices 614 are envisioned for use in the holding device 610.

The foregoing description has been presented for purposes of illustration and description. It is not intended to be exhaustive or to limit the invention to the precise forms disclosed. Obvious modifications or variations are possible in light of the above teachings. The embodiment discussed, however, was chosen and described to provide the best illustration of the principles of the invention and its practical application to thereby enable one of ordinary skill in the art to utilize the invention in various embodiments and with various modifications as are suited to the particular use contemplated. All such modifications and variations are within the scope of the invention as determined by the appended claims when interpreted in accordance with the breadth to which they are fairly and legally entitled.

1. A system for facilitating bone grafting, comprising:
a rasp configured to prepare a grafting surface of a recipient;
a sizer configured to determine a measurement of a graft for abutment with the grafting surface;
a cutting guide configured to receive a bone stock and to facilitate producing said graft in proportion to said sizer;
a measuring pan configured to determine an amount of grafting substance required to fill a void in said graft; and
a holding device configured to stabilize said graft as the grafting substance is placed in said graft.

2. The system as claimed in claim 1, said rasp further comprising:
a head having a first surface, a second surface, and an intermediate surface disposed therebetween and defined by a periphery of said first surface and a periphery of said second surface; and
a handle attached to said intermediate surface configured for ease of using said rasp.

3. The system as claimed in claim 2, said intermediate surface being continuous and smooth.

4. The system as claimed in claim 3, said first and said second surfaces being parallel and having serations disposed thereon for preparing the grafting surface.

5. The system as claimed in claim 3, wherein said first and said second surfaces, each lying in a plane, define an angle therebetween.

6. The system as claimed in claim 5, said handle being disposed along an axis defining the greatest amount of separation between corresponding positions on the peripheries of said first surface and said second surface.

7. The system as claimed in claim 6, said angle being approximately 7 degrees.

8. The system as claimed in claim 7, said handle being normal to said intermediate surface.

9. The system as claimed in claim 1, said sizers further comprising:
a body having a primary surface, a secondary surface, and a mid surface disposed therebetween, said mid surface being continuous and defined by a periphery of said first surface and a periphery of said second surface; and
a member attached to said mid surface and configured such that said sizes may be used without handling said body.

10. The system as claimed in claim 9, said member being normal to said mid surface.

11. The system as claimed in claim 9, said primary and secondary surfaces being parallel and smooth.

12. The system as claimed in claim 9, said primary and secondary surfaces each lying in a plane, the planes defining an angle therebetween.

13. The system as claimed in claim 12, said handle being disposed along an axis defining the greatest amount of separation between corresponding positions on the peripheries of said primary surface and said secondary surface.

14. The system as claimed in claim 12, said angle being approximately 7 degrees.

15. The system as claimed in claim 1, said cutting guide further comprising:
a base having a longitudinal axis and configured for mounting a guide structure;
a first guide structure and a second guide structure being mounted to said base, said first guide structure and said second guide structure having a plurality of guide slots formed therein; and
wherein said first guide structure, said second guide structure, and said base define a cavity configured to receive a bone stock from which said graft is produced.

16. The system as claimed in claim 15, wherein said first guide structure and said second guide structure further comprise planar members.

17. The system as claimed in claim 16, said first guide structure and said second guide structure being parallel and mounted to said base such that they are parallel to said longitudinal axis and normal to said base.

18. The system as claimed in claim 16, wherein said first guide structure and said second guide structure are configured to receive a top, such that said base, said first guide structure, said second guide structure, and said top define an enclosure configured to receive the bone stock, wherein the plurality of guide slots is similarly formed in said top.

19. The system as claimed in claim 17, wherein said guide slot is configured such that a line defined by the intersection of a plane containing said guide slot and a plane containing said longitudinal axis forms a right angle with said longitudinal axis.

20. The system as claimed in claim 19, wherein a first plane contains a first guide slot and a second plane contains said second guide slot, said first plane and said second plane defining an angle therebetween.

21. The system as claimed in claim 20, wherein said angle is approximately 7 degrees.

22. The system as claimed in claim 1, said measuring pan further comprising a planar surface having a plurality of cups formed therein.

23. The system as claimed in claim 22, wherein said cups differ in volume from each other.

24. The system as claimed in claim 22, each of said cups forming a cylindrical volume.

25. The system as claimed in claim 24, said cup further comprising a cylindrical side wall and a bottom.

26. The system as claimed in claim 24, wherein said plurality of cups is disposed between said planar surface and a second plane, said second plane and said planar surface being parallel.

27. The system as claimed in claim 1, said holding device further comprising:
a planar support configured for receiving the graft and for mounting an adjustment device; and
an adjustment device mounted to said planar support and configured to secure the graft, wherein the graft can vary in size.

28. The system as claimed in claim 27, said adjustment device further including:
an abutment surface configured to abut the graft;
an adjustment support including a passage therethrough, mounted to said planar support, said passage configured to receive an adjustment member; and
an adjustment member having a longitudinal axis, a proximal end, and a distal end, said adjustment member passing through said passage and configured for motion along said longitudinal axis.

29. The system as claimed in claim 27, wherein a plurality of said adjustment devices are mounted to said planar support.

30. The system as claimed in claim 28, wherein said abutment surface further comprises said proximal end of said adjustment member, said proximal end being configured to abut the graft.

31. The system as claimed in claim 28, wherein said abutment surface further comprising a plate rotatably attached to said proximal end of said adjustment member.

32. The system as claimed in claim 27, said holding device further comprising an abutment member mounted on said planar support and configured to receive the graft.

33. The system as claimed in claim 32, wherein said abutment member is a V-shaped wall having an inner surface and an outer surface, positioned on said planar support such that said inner surface opposes said adjustment device.

34. A method of producing a graft, comprising the steps of:
determining the dimensions of a graft;
providing a cutting guide configured to receive a bone stock and including a plurality of guide slots formed therein;
inserting the bone stock into said cutting guide;
securing the bone stock in said cutting guide;
cutting the graft from the bone stock; and
wherein said guide slots are configured such that the graft will have the desired dimensions after said cutting step.

35. The method as claimed in claim 34, wherein said step of determining the dimensions further includes placing a sizer between a first segment and a second segment to be fused.

36. The method as claimed in claim 35, further comprising the steps of:
securing the graft for packing with a grafting substance; and
measuring amount of the grafting substance during packing of the graft.

37. The method as claimed in claim 36, said step of securing further comprising placing graft in abutment with an abutment member and adjusting an adjustment device such that the graft is secured therebetween.

38. The method as claimed in claim 36, said measuring step further comprising placing the grafting substance in a cup of known volume prior to packing the graft.
